(19) United States
(12) Patent Application Publication
(10) Pub. No.: US 2004/0176728 A1
(43) Pub. Date: Sep. 9, 2004

(54) Cartridge-free, multi-dose injection apparatus

(76) Inventors: Mark James Fisher, Highland Park, IL (US); Robert Nesbitt, Fishers, IN (US)

Correspondence Address: ELI LILLY AND COMPANY, PATENT DIVISION, P.O. BOX 6288, INDIANAPOLIS, IN, 46206-6288, US

(21) Appl. No.: 10/483,243

(22) PCT Filed: Jul. 15, 2002
(86) PCT No.: PCT/US02/19814

Related U.S. Application Data
(60) Provisional application No. 60/305,733, filed on Jul. 16, 2001.

(51) Int. Cl. A61M 5/00

(52) U.S. Cl. 604/191

(57) ABSTRACT

A prefilled injection apparatus for multiple dosings of medication. The protective housing element into which is installed the mechanical drive mechanism used to force the medication from the apparatus extends forward to provide a volume in which is directly contained a multi-dose quantity of medicine, and the medicine is sealed between a movable piston and a septum each in fluid tight engagement with the protective housing element. This design advantageously eliminates the need for using a separate cartridge within the apparatus. A method of making a cartridge-free, multi-dose injection apparatus is also disclosed.

### BACKGROUND OF THE INVENTION

The present invention pertains to medication dispensing devices, and, in particular, to a portable injection apparatus such as an injection pen.

Patients suffering from a number of different diseases frequently must inject themselves with medication. To allow a person to conveniently and accurately self-administer medicine, a variety of injecting devices broadly known as injector pens or injection pens have been developed. Typically, these pens hold a cartridge having a glass housing and including a piston and containing a multi-dose quantity of liquid medication. A drive member, extending from within a base of the injection pen and operably connected with typically more rearward mechanisms of the pen that control drive member motion, is movable forward to advance the piston in the cartridge in such a manner to dispense the contained medication from an outlet at the opposite cartridge end, typically through a needle that penetrates a stopper at that opposite end. In disposable pens, which are also known as prefilled pens, after a pen has been utilized to exhaust the supply of medication within the cartridge, the entire pen is discarded by a user, who then begins using a new replacement pen.

While useful, cartridge-based injection devices are not without their shortcomings. For example, in prefilled pens, the assembly of a separate glass-housing cartridge into a protective pen housing not only involves a step in the manufacturing process, but also increases the dimensions as well as weight of the device.

In a disclosed injection pen system that does not employ a separate glass cartridge within a protective housing, a plastic cartridge is provided that attaches directly to the end of the housing in which the drive mechanism of the device is housed. While this alternate system may be advantageous in some respects, the number of component parts requiring manufacture and assembly during production still may be greater than desirable.

Thus, it would be desirable to provide an apparatus that can overcome one or more of these and other shortcomings of the prior art.

### BRIEF SUMMARY OF THE INVENTION

The present invention encompasses a prefilled injection apparatus for multiple dosings of medication, which apparatus integrates conventional cartridge features into an extension of the protective housing of the apparatus, which housing holds the mechanical drive mechanism used to force the medication from the apparatus, thereby eliminating the need for using a separate cartridge.

In one form thereof, the present invention provides a cartridge-free, multi-dose injection apparatus including an external housing including a tubular body, the external housing body defining a hollow interior having a proximal end and a distal end, a multi-dose quantity of medicine within a distal end portion of the external housing and sealed between a movable piston and a septum, a drive member extending within the hollow interior for advancing the piston, a manual actuator external to the housing, and an injecting assembly extending within a proximal end portion of the external housing and operably connected with the actuator, the injecting assembly being operable by shifting the actuator from a first position to a second position to advance the drive member to shift the piston distally for dispensing a dose of the medicine through an opening in the septum. The external housing body, along at least a portion of its periphery, has a one-piece plastic construction extending from the proximal end to the distal end. The piston slides within the hollow interior in a fluid tight engagement with an internal surface of the external housing body. At least a majority of a length of the injecting assembly fits within the hollow interior of the external housing body at least when the actuator is disposed in the second position.

In another form thereof, the present invention provides a cartridge-free, multi-dose injection apparatus including a tubular external housing body formed from plastic with a one-piece construction, the external housing body defining a hollow interior having a proximal end and a distal end, a multi-dose quantity of medicine within a distal end portion of the external housing body hollow interior and sealed between a movable piston and a septum each in fluid tight engagement with the external housing body, a drive member extending within the hollow interior for advancing the piston, a manual actuator external to the housing body, and an injecting assembly extending within a proximal end portion of the external housing body and operably connected with the actuator, the injecting assembly being operable by shifting the actuator from a first position to a second position to advance the drive member to shift the piston distally for dispensing a dose of the medicine through an opening in the septum.

In still another form thereof, the present invention provides a method of making a cartridge-free, multi-dose injection apparatus, including the steps of: forming from plastic an external housing having a tubular body, the tubular housing body having a distal end and a proximal end, the tubular body having an interior surface defining a hollow interior; installing a first sealing member that sealingly engages the tubular housing body either at the distal end of the tubular housing body or at a point within the hollow interior and along the tubular housing body length between the distal and proximal ends; filling a distal portion of the tubular housing body hollow interior with a multi-dose quantity of medicine; after the filling of the medicine, installing a second sealing member that sealingly engages the tubular housing body at the other of the distal end of the tubular housing body and the point within the hollow interior and along the tubular housing body length between the distal and proximal ends, whereby the multi-dose quantity of medicine is sealed directly within the tubular housing body between the first and second sealing members; and installing an injecting assembly and a drive member in a distal portion of the tubular housing body hollow interior after the filling of the medicine, the injecting assembly operable by an actuator external to the hollow interior to advance the drive member to shift one of the first and second sealing members within the tubular housing body toward the other of the first and second sealing members for dispensing a dose of the medicine.

One advantage of the present invention is that a medication injection apparatus can be provided which may facilitate production by reducing the number of component parts requiring manufacture and assembly.

Another advantage of the present invention is that a medication injection apparatus can be provided which is slimmer and lighter weight in design than some other injection apparatuses.

Still another advantage of the present invention is that a medication injection apparatus can be provided with a simpler design that may reduce overall costs.

Yet another advantage of the present invention is that a medication injection apparatus can be provided in which the drug-containing part is injection molded from plastic with tight tolerances, resulting in the apparatus likely being more predictable in terms of certain operational characteristics, such as force required to eject the medication therefrom.

Another advantage of the present invention is that a medication injection apparatus can be provided which, due to the use of a plastic medicine container and the reduction of parts, may improve dose accuracy.

### BRIEF DESCRIPTION OF THE DRAWINGS

The above-mentioned and other advantages and objects of this invention, and the manner of attaining them, will become more apparent, and the invention itself will be better understood by reference to the following description of embodiments of the invention taking in conjunction with the accompanying drawings, wherein:

FIG. 1 is a front elevational view of a first embodiment of a medication injection apparatus of the present invention;

FIG. 2 is a longitudinal cross-sectional view of the medication injection apparatus of FIG. 1 prior to the mounting of a needle assembly, and wherein an actuator, a drive member, and an injecting assembly that converts actuator input into drive member advancement are diagrammatically shown;

FIG. 3 is a longitudinal cross-sectional view of the medication injection apparatus of FIG. 1 at an early stage of its construction;

FIG. 4 is a view similar to the view of FIG. 3, at a later stage of construction; and

FIG. 5 is a view similar to the view of FIG. 4 at a later stage of construction.

Corresponding reference characters indicate corresponding parts throughout the several views. Although the drawings represent an embodiment of the present invention, the drawings are not necessarily to scale, and certain features may be exaggerated or omitted in some of the drawings in order to better illustrate and explain the present invention.

### DETAILED DESCRIPTION OF THE INVENTION

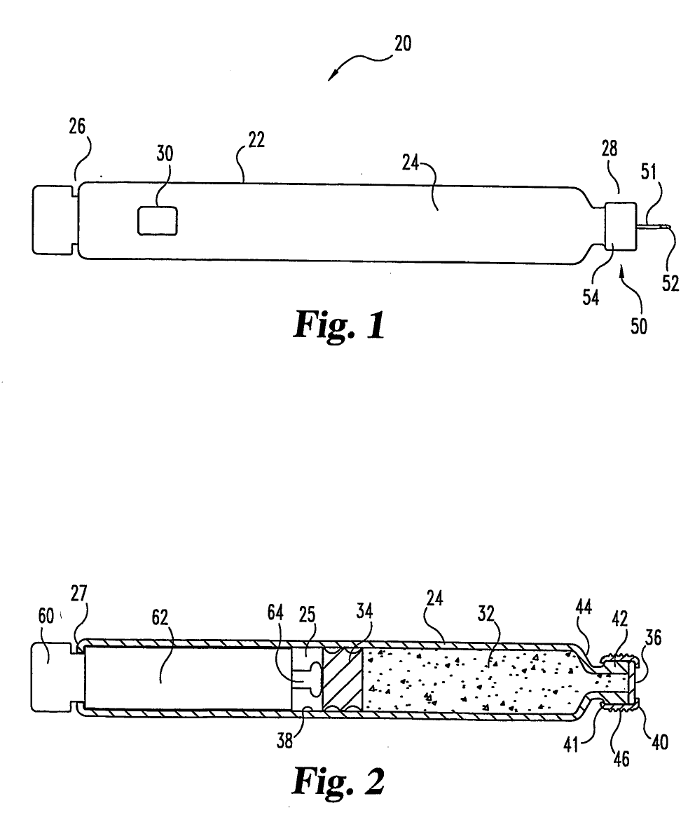

Referring now to FIGS. 1 and 2, there is shown a first embodiment of a cartridge-free, multi-dose medication injection apparatus of the present invention. Any directional references in this detailed description with the Figures, such as up or down, are intended for convenience of description, and by itself does not limit the present invention or any of its components to any particular positional or spatial orientation.

The apparatus, generally designated 20, is shown as an injection pen, which pen has an elongated, substantially writing instrument-like form, although other forms are within the scope of the invention. Medication injection pen 20 is a prefilled or disposable pen, in that after the quantity of medicine contained therein is exhausted by multiple operations of the pen, the entire pen is discarded.

Injection pen 20 includes an external, protective housing 22. In the shown embodiment, the housing is provided entirely by a cylindrical tubular body 24 extending from a proximal end 26 to a distal end 28. Body 24 is formed in one-piece, such as by injection molding, out of a polymeric or plastic material that is suitable for use with the medicine it directly contacts and holds as described below. Body 24 may be transparent to allow the contained medicine to be visible to let a user estimate the amount remaining, and the portion surrounding the pen mechanics may have label materials molded therein. Opening 30 in body 24, which may be covered by a magnifying lens, allows for a dose display to be visible to a user. In an alternate embodiment, body 24 may be fashioned in a similar shape from a pair of mating, longitudinally extending pieces that before medicine filling are fixedly secured together, such as by ultrasonic welding or adhesives, but a one-piece body construction is preferred for minimizing parts needed to be manufactured and assembled. Although external housing 22 consists of body 24 in the shown embodiment, one or more additional housing parts, such as an annular cap for the proximal end of the pen body if desired, may also be employed within the scope of the invention.

Sealed within the hollow interior 25 of tubular body 24 at the distal portion of the housing 22 is a multi-dose quantity of a fluid medicine 32 to be delivered by operations of pen 20. Medicine 32 may be any of a variety of drug products, such as diabetes medicines such as insulins, which do not react with the housing. A quantity of 1.5 ml or 3.0 ml of insulin provides for at least several maximum doses of the typical injection pen, and many multiples thereof for smaller dose amounts. Medicine 32 is sealed directly within housing body 24 between a movable piston 34 and a stopper or septum 36. Piston 34, which may be made of an elastomeric material, axially slides within hollow interior 25 along a middle section of the housing body length. Piston 34 has a sealing periphery in a fluid tight engagement with the hollow interior-defining internal surface or wall 38 of housing body 24 to contain the fluid medication.

Septum 36 is made of an elastomeric material and serves to seals against external housing body 24 to cover the distal, outlet end of hollow interior 25 and thereby contain medication 32. Septum 36 is held in place by an apertured cap 40 fit thereover and secured to housing body 24. In the shown embodiment, cap 40 is a made of metal and is secured via a depending collar 41 crimped over a radially outwardly projecting annular lip 42 integrally formed on a stepped-down diameter neck region 44 of the housing body. In alternate embodiments, the cap may be otherwise formed, such as made of plastic with its depending collar staked or otherwise secured to housing body 24.

The exterior surface of collar 41 is integrally provided with at least one attachment module indicated at 46 adapted to removably mount a disposable needle assembly, generally designated 50, shown in FIG. 1. Module 46 is shown as a continuous encircling thread formed in collar 41, but other suitable connection means may be provided.

Needle assembly 50 is of known design and includes a double-ended needle cannula or injection needle 51 having a distal tip 52 at one end and a not shown proximal point at the other. Injection needle 51 is mounted in a tubular hub 54 that is screwable onto and off of cap threading 46. Although the needle assembly is shown as having a single injection needle, needle assemblies which may be used with pen 20 may be of various types known in the art, including, but not limited to, assemblies with one or more shortened injection needles, including microneedle arrays.

When needle assembly 50 is mounted on threading 46 as shown in FIG. 1, the proximal point of injection needle 51 extends through the aperture of cap 40, and penetrates and thereby provides an opening in cartridge septum 36 to provide a fluid flow outlet by which medicine within hollow interior 25 can be dispensed through needle 51 when piston 34 is moved toward the needle assembly 50 in a conventional manner during injecting use of pen 20.

Positioned proximally of piston 34 are the components that function, in a preferred embodiment, to allow any one of a number of quantities of medicine to be selected and then expelled from the pen by a user. As further diagrammatically represented in FIG. 2, these components include a manually operable actuator in the form of a dosage knob 60 that projects from the proximal end of pen housing 22, a mechanical drive assembly 62 operably connected to knob 60 and which extends within the proximal end portion of the housing body 24, and a drive member 64 extending in an axial or longitudinal direction within hollow interior 25 and abutting piston 34. Depending on the workings of drive assembly 62, drive member 64 may be, for example, a rotating screw or a rotatably fixed, axially shiftable toothed shaft.

Dosage knob 60 is rotatable in a conventional manner to set the dose to be injected, which rotation causes knob 60 to move to an extended position farther from the proximal end of the housing. When knob 60 is then axially plunged toward the pen housing from this extended first position to a second position shown in FIGS. 1 and 2, the drive assembly 62 operates to cause drive member 64 to advance in the distal direction to shift piston 34 distally for dispensing the set dose of the medicine through needle 51. In this shown embodiment, and while particular pieces of the drive assembly 62 may axially move during use, the drive assembly overall is axially secured within the volume indicated at 62 in FIG. 2. Such securement may be achieved by attaching, such as via ultrasonic welding, the drive assembly to the interior wall 38 of tubular body 24, or by mechanically capturing the drive assembly, such as by using a cap at the proximal end of the housing or forming body 24 with an inwardly extending flange 27.

Although dosage knob 60 moves out relative to pen housing 22 when rotated to increase the set dose as described above, and then moves in relative to the pen housing when rotated to decrease a dose set too large, such axial motion during dose setting is a function of the mechanical workings of the pen, and the dosage knob 60 need not so move to fall within the scope of the invention. For example, the dosage knob may be axially fixed when rotated to set the dose, and then plunged farther into the pen housing to inject a dose. In addition, while the element actuated to cause the drive assembly to advance drive member 64 and thereby piston 34 distally is described herein as the same element or knob used to set the dose, the actuating element may be different from the dose setting element within the scope of the invention.

The foregoing general description of the mechanism used to advance piston 34 is intended to be illustrative and not limiting in any way. A variety of injection pens, including pull to prepare/push to inject type injection pens, are known in the art which include different mechanisms that allow setting and administering doses, and the mechanisms may be readily adapted for use in a cartridge-free injection pen as described herein. Still further, if pen 20 were intended to always deliver the same or a fixed dose, the mechanism could be configured to appropriately advance piston 34 to achieve such fixed dose upon each activation of the external actuator.

The entire axial length of drive assembly 62 is protectively housed within tubular body 24 at all times of pen use in the shown embodiment. Lesser length portions of a drive assembly, such as substantially all or a majority of such length, also can fit within hollow interior 25 of external housing body 24 in alternate embodiments of the present invention. Moreover, such fitting within hollow interior 25 need not be at all times of use, as it is within the scope of the invention for part of the drive assembly to be caused to temporarily project farther beyond the proximal end of the housing during, for example, dose setting.

The structure of pen 20 will be further understood in view of the following explanation of one manner of its construction. A clean room environment and sterilizing and the like will naturally be employed through the production process as appropriate to provide a suitable final product.

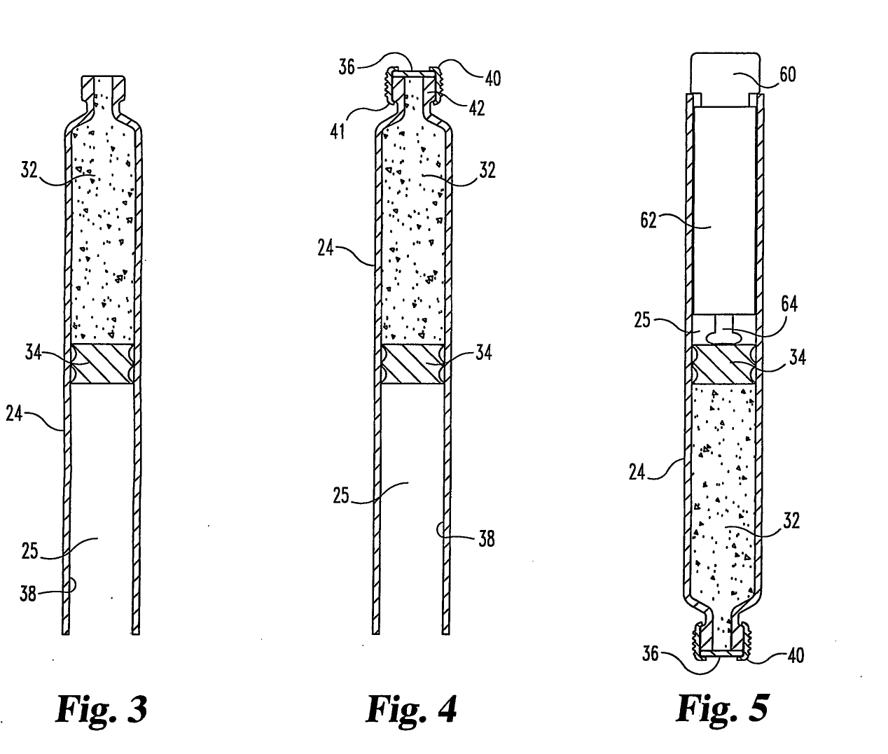

After the external housing 22 is formed from plastic in one-piece by injection molding, piston 34 is installed within hollow interior 25 so as to sealingly engage housing body 24 along a central portion of the body length. With the housing distal end pointing upward, the distal portion of the tubular housing body hollow interior 25 is then filled with a multi-dose quantity of medicine 32. At this stage of the manufacture, the in-progress pen is arranged as shown in FIG. 3.

Next, septum 36 is secured in sealing engagement with the tubular housing body 24 by the crimping of cap 40 onto body lip 42, such that the multi-dose quantity of medicine 32 is now sealed directly within the tubular housing body 24 between piston 34 and septum 36. At this stage of the manufacture, the in-progress pen is arranged as shown in FIG. 4. It will be appreciated that an alternate manufacturing sequence involves sequentially securing the septum, filling the medicine, and then inserting the piston.

The medicine-filled pen housing is then inverted, and the preassembled mechanics of the pen are inserted into the proximal portion of the tubular housing body hollow interior 25, leaving the actuator 60 projecting from the hollow interior. The in-progress pen is now arranged as shown in FIG. 5. After securing the drive assembly 62 to the housing body 24, such as via ultrasonic welding and/or inturned flange 27, the manufacturing is complete, and pen 20 is arranged as shown in FIG. 2.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

We claim:

1. A cartridge-free, multi-dose injection apparatus comprising
an external housing including a tubular body, said external housing body defining a hollow interior having a proximal end and a distal end;
a multi-dose quantity of medicine within a distal end portion of said external housing and sealed between a movable piston and a septum;
a drive member extending within said hollow interior for abutting said piston;
a manual actuator external to said housing;
an injecting assembly extending within a proximal end portion of said e housing and operably connected with said actuator, said injecting assembly operable by shifting said actuator from a first position to a second position to advance said drive member to shift said piston distally for dispensing a dose of the medicine through an opening in said septum;
wherein said external housing body, along at least a portion of its periphery, has a one-piece plastic construction extending from said proximal end to said distal end;
wherein said piston slides within said hollow interior in a fluid tight engagement with an internal surface of said external housing body, and
wherein at least a majority of a length of said injecting assembly fits within said hollow interior of said external housing body at least when said actuator is disposed in said second position.

2. The cartridge-free, multi-dose, injection apparatus of claim 1 wherein at least substantially all of the length of said injecting assembly is disposed within said hollow interior of said external housing body.

3. The cartridge-free, multi-dose injection apparatus of claim 1 wherein all of the length of said injecting assembly is disposed within said hollow interior of said external housing body.

4. The cartridge-free, multi-dose injection apparatus of claim 1 wherein the opening in said septum is provided by a penetrating needle of a disposable needle assembly removably mounted at a distal end of said external housing.

5. The cartridge-free, multi-dose injection apparatus of claim 4 wherein said septum is sealingly mounted to said external housing body to cover said distal end of said hollow interior of said external housing body.

6. The cartridge-free, multi-dose injection apparatus of claim 5 wherein said septum is sealingly mounting to said external housing body by an apertured cap fit over said septum and secured onto said housing body, said cap further comprising at least one attachment module provided on an exterior surface, said at least one attachment module adapted to removably mount the disposable needle assembly.

7. The cartridge-free, multi-dose injection apparatus of claim 6 wherein said cap is crimped onto said housing body.

8. The cartridge-free, multi-dose injection apparatus of claim 1 wherein the entire external housing body is molded as a single piece.

9. A cartridge-free, multi-dose injection apparatus comprising:
a tubular external housing body formed from plastic with a one-piece construction, said external housing body defining a hollow interior having a proximal end and a distal end;
a multi-dose quantity of medicine within a distal end portion of said external housing body hollow interior and sealed between a movable piston and a septum each in fluid tight engagement with said external housing body;
a drive member extending within said hollow interior for advancing said piston;
a manual actuator external to said housing body;
an injecting assembly extending within a proximal end portion of said external housing body and operably connected with said actuator, said injecting assembly operable by shifting said actuator from a first position to a second position to advance said drive member to shift said piston distally for dispensing a dose of the medicine through an opening in said septum.

10. A method of making a cartridge-free, multi-dose injection apparatus, comprising the steps of:
forming from plastic an external housing having a tubular body, said tubular housing body having a distal end and a proximal end, said tubular body having an interior surface defining a hollow interior;
installing a first sealing member that sealingly engages said tubular housing body either at the distal end of said tubular housing body or at a point within the hollow interior and along the tubular housing body length between the distal and proximal ends;
filling a distal portion of the tubular housing body hollow interior with a multi-dose quantity of medicine;
after the filling of the medicine, installing a second sealing member that sealingly engages said tubular housing body at the other of said distal end of said tubular housing body and the point within the hollow interior and along the tubular housing body length between the distal and proximal ends, whereby said multi-dose quantity of medicine is sealed directly within said tubular housing body between said first and second sealing members; and
installing an injecting assembly and a drive member in a distal portion of the tubular housing body hollow interior after the filling of the medicine, said injecting assembly operable by an actuator external to said hollow interior to advance said drive member to shift one of said first and second sealing members within said tubular housing body toward the other of said first and second sealing members for dispensing a dose of the medicine.

11. The method of claim 10 wherein said first sealing member comprises a piston that sealingly engages said tubular housing body at the point within the hollow interior and along the tubular housing body length between the distal and proximal ends, said piston shiftable distally by advancement of said drive member.

12. The method of claim 11 wherein said step of installing said second sealing member comprises crimping an apertured cap onto said distal end of said tubular housing body over a septum covering an end of said hollow interior.

13. The method of claim 12 further comprising the step of mounting a needle assembly to at least one attachment module provided on the exterior of said apertured cap.

14. The method of claim 10 wherein said tubular housing body is molded from plastic as a single piece.
